SEQUENCE LISTING (5 sequences)

SEQ ID NO: 1 - DNA, 1500 bp, Artificial Sequence
  sample artificial sequence pattern used as an example
gccgctaccgccgtaggtgaagcgtcgactgcgcgaacgcgagaaccgggcgagggtgaggggctacgagggcagctcgacggggctgcccgcattacgtggaggtccgcaagtgaaatggaatgtcgcgagtagaagggtggtgccctatgtcgaagtgcgcttggcgcatggctacattaagcggagcacttcctggccacgctgcgtgtctgatgcaactttgcgtctcactgatgcagagtctaacgccgctgtttgacccgcgtaaattactttatccatcgcgcctttcggcggctgattaatcaccccaacccgcagatcagtaacttttgagaaaccgtgcccggtgtttgaaatcctccagttaactcggacccattatccgtcgtgcgctaagtttccatacgtcatccccgggcatgttgcgccgctataattgggacgtgaagtaggcaaacagacagagaaccaccgtatttcgagtcagcctccgtgaaaatgatcgaaaggccgacgagaaaactaggctgaaggaccaacgaatcgtttccgcgggctcgtgttttacacaaccgttcttctttagctacccgacggtcaaacacaacctccagtcatgcagtcgaaagaacatgcgaggtttgttgactcgttccgccgtgcgtccagacgtcagtggacggatgttagatggcgacaactacctgatatccgtgtgaacaatcatcggctcagacttgccgccttgactctcaggttcaataacttgcagatagggcagcgcggggtgtgctctcgcagcacacatattgccagataccacgacatcagtgactggttagacggcgccagatgtgacagcatactaagggcgtgctgacaccgcgaaaactccctctctaaagggactatctaagggacgtgaacatcggaggttggagcacgcacttaatgcagtgcgttccagagcagcagtcggattaatagctccgtttacaagctctgtacgcatgtaacatgtcgcaaccaccacaggtaccgctaccatgtctcctttaactcagcgaaactataggttcgcccctgaattcaagtgcgcccagtgatcctgggtcgcggtatccgagcgggaaattaatgctagcgaggatcataactagttcggtaaacctgtaataacttttcgatttcctgcatacaagtactgctgcagcataatcgggatgttactccgaagttaagcgcccaactaggcagtttggcagcatgacgagccagcagcacccggcattcttacccagctcgaatcgcatctggagggagtcaggcctttccaaacagcagtagttgaagtcctttcaagatcctccaggtgtctctgaaacatttaaggcaaatcacactatacaaattcgcgcgaaccgaccaatgtctccctagactaggtccgttacgtcccgctttacatttgatcagggctgccccttgaaatga

SEQ ID NO: 2 - DNA, 12 bp, Artificial Sequence
  sample artificial sequence pattern used as an example
cgttcgatctga

SEQ ID NO: 3 - DNA, 36 bp, Artificial Sequence
  sample artificial sequence pattern used as an example
cccgggttttttcccgggaaatttccctttgggaaa

SEQ ID NO: 4 - DNA, 36 bp, Artificial Sequence
  sample artificial sequence pattern used as an example
atggaccagccacaagcgctactgttccgacgtcta

SEQ ID NO: 5 - DNA, 36 bp, Artificial Sequence
  Sample artificial sequence pattern used as an example
cgtagtgtcggtaccatactagacggagcttaccta

(19) United States
(12) Patent Application Publication
(10) Pub. No.: US 2020/0152293 A1
(43) Pub. Date: May 14, 2020

(54) SYSTEM AND METHOD FOR APPLICATION OF ENHANCED CONTROLS WITH A GENOMIC COMPUTING PLATFORM

(71) Applicant: Reference Genomics, Inc., San Francisco, CA (US)

(72) Inventors: Roderick Jock Bovee, South San Francisco, CA (US); Nickolas Boyd Greenfield, San Francisco, CA (US)

(21) Appl. No.: 16/600,226

(22) Filed: Oct. 11, 2019

Related U.S. Application Data
(60) Provisional application No. 62/744,412, filed on Oct. 11, 2018.

(51) Int. Cl. G16B 30/10; G16B 50/20; G16B 40/10; G16B 25/10; G06F 9/30; G16B 50/40; G06F 17/18; G16B 50/30; C12Q 1/6869; G16B 50/50; H03M 13/19

(52) U.S. Cl. CPC G16B 30/10; G16B 50/20; G16B 40/10; G16B 25/10; G06F 9/30029; G16B 50/40; C12Q 2500/00; G16B 50/30; C12Q 1/6869; G16B 50/50; H03M 13/19; C12Q 2600/166; G06F 17/18

(57) ABSTRACT

A system and method for genomic sample processing in a computing system using an enhanced spike-in that includes specifying a genetic control sequence; registering the genetic control sequence to a genetic database of the computing system; in association with a genomic sample, detecting a detected instance of the control sequence in a collection of genetic data collected for the genomic sample; and augmenting sample management of the genomic sample within the computing system according to the detected instance of the control sequence.

### CROSS-REFERENCE TO RELATED APPLICATIONS

This Application claims the benefit of U.S. Provisional Application No. 62/744,412, filed on 11 Oct. 2018, which is incorporated in its entirety by this reference.

### TECHNICAL FIELD

This invention relates generally to the field of genomic sequencing, and more specifically to a new and useful system and method for application of enhanced controls with a genomic computing platform.

### BACKGROUND

Since the mid 1900s, we have had knowledge of DNA as the building block of life. DNA, only consisting of four building blocks, the four base nucleotides: thymine (T), adenine (T), cytosine (C), and guanine (G); that just by the ordering of these four nucleotides gives way to complex and long sequences that describe nearly the entire diversity of life. With this knowledge, there has been a push to understand each piece of this building block to finer detail and to understand our own unique construction from these building blocks.

The field of DNA sequencing finally started taking off in the 1970s with the sequencing of short DNA strands using techniques of identifying single nucleotides, one at a time. As the field of DNA sequencing progressed and improved, a culmination was reached with the start and completion of the human genome project (1990-2003), wherein the entire genetic sequence of one human was sequenced through a worldwide effort. Throughout this time there was a huge push to develop faster and cheaper techniques for DNA sequencing. As techniques improved, there was a change from single base sequencing techniques to “shotgun” methods that entailed chopping DNA strands into large chunks, sequencing the chunks, and then recombining them.

Since the human genome project and the privatization of DNA sequencing, there has been an even greater push towards cheaper, faster, and more accurate sequencing techniques. These techniques, often referred to as next generation sequencing, have moved even more into the realm of automated and high throughput analyses of fragments of DNA that are then recombined using large amounts of computational power.

Although significant innovation has taken place along the lines of automation of high throughput DNA sequencing, not as much innovation has taken place along the lines of experimental controls. Cross contamination and other sample handling issues can impact sequencing results, which can be especially true for metagenomics applications—the study of genetic material from environmental or other complex samples including rich microbial communities—where the detection and accurate quantitation of low abundance organisms can be critically important. Thus, there is a need in the genomic sequencing field to create a new and useful system and method for application of enhanced controls with a genomic computing platform. This invention provides such a new and useful system and method.

### BRIEF DESCRIPTION OF THE FIGURES

FIGS. 1 and 2 are schematic representations of systems of preferred embodiment;

FIG. 3 is an exemplary representation of an output of a control sequence generator engine;

FIGS. 4A and 4B are exemplary screenshots of views of a genomic computing platform using modifying presentation of sample analysis based on detection of DNA control sequence;

FIG. 5 is a flowchart of a method of preferred embodiment;

FIGS. 6 and 7 are flowcharts of exemplary encoding and decoding approaches;

FIG. 8 is block diagram of generating a control sequence from a control sequence generation engine;

FIG. 9 is a block diagram of decoding a detected control sequence with control sequence processor;

FIGS. 10 and 11 are flowchart representations of alternative method variations;

FIG. 12 is a flowchart representation of variations for augmenting sample management

FIG. 13 is block diagram of generating an exemplary control sequence; and

FIG. 14 is a block diagram of decoding an exemplary detected control sequence.

### DESCRIPTION OF THE EMBODIMENTS

The following description of the embodiments of the invention is not intended to limit the invention to these embodiments but rather to enable a person skilled in the art to make and use this invention.

### 1. Overview

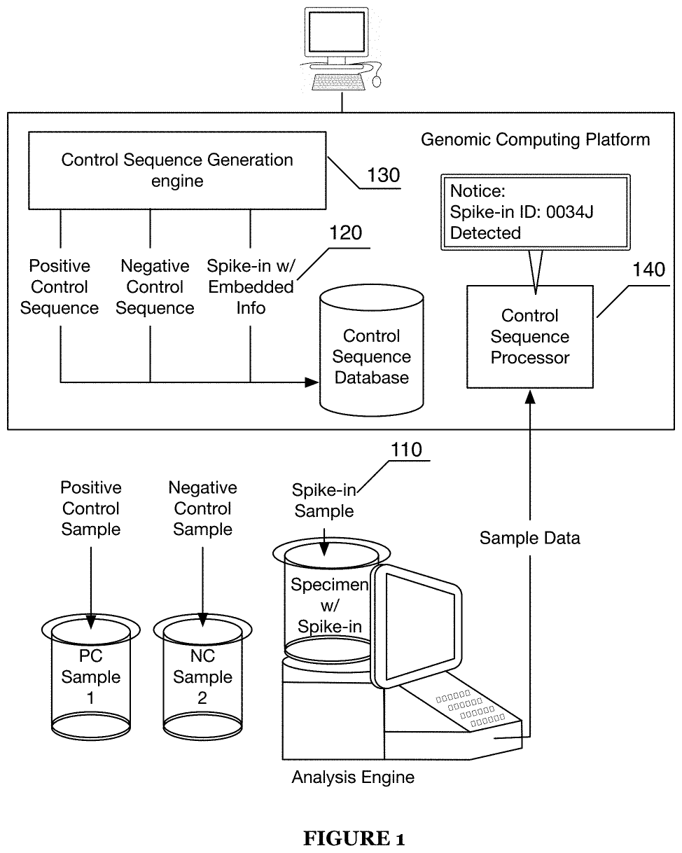

As shown in FIG. 1, a system and method for application of enhanced controls with a genomic computing platform functions to implement automatic control management within a computer implemented software system. The system and method preferably provide a mechanism for linking or registering experimental controls during genetic sample processing and then automating the software analysis of that processing using information of the controls. In some preferred variations, automatic detection of a type of control sample can initiate automatic analysis within a genomic computing platform.

The system and method may be used with one or multiple various types of control samples. The control samples of the system and method may be dynamically detected and used for purposes of internal controls, positive controls, and/or negative controls as shown in FIG. 1. Automated control sample management in a genomic computing platform may make use of registered control samples with embedded information and/or with externally stored information. Different variations of the system and method may make use of synthetic and/or organic control samples.

In one preferred variation of the system and method, the system and method can be used for the application of an enhanced spike-in with a genomic computing platform, which functions to use a genetic control sample with embedded metadata to augment genomic and sequencing workflows. A synthetic control sample with embedded information may be used as an internal control and/or used in a variety of other ways.

In another alternative variation of the system and method, the system and method can be can be used with other synthetic or organic control samples. Organic control samples in particular may be selected and registered as positive control samples such that upon detecting and classifying the sample, the registration of that control sample as a positive control can trigger the genomic computing platform to evaluate the associated sequencing data as a positive control.

Herein, the generation, use, and application of a synthetic enhanced spike-in with embedded information are used as a primary example in describing the system and method. However, the system and method may additionally or alternatively be used with additional control samples that can trigger automatic analysis and processing within a computer implemented software system.

The system and method can preferably be used to enable accurate and automated control of DNA (Deoxyribonucleic acid) assays or RNA (ribonucleic acid) assays, in particular with relation to DNA or RNA sequencing assays.

The enhanced spike-in of the system and method is preferably a genetic control sample that encodes metadata or information such that the control sample may be auto-descriptive upon detection. That is to say, that detection of the spike-in can enable the extraction of the embedded information in a reliable manner. An enhanced spike-in may be used in various experiments and workflows, where the spike-in can serve as a physical, detectable communication of information to a processing systems or devices. The system and method described herein prescribe an approach to design, analysis, validation, and/or application of error-correcting internal spike-in controls for metagenomics. The system and method are preferably applied to use of a DNA control sequence, but may alternatively be applied to RNA (Ribonucleic acid) or other suitable genetic biological sequence controls.

Herein, reference to a genetic or genomic sample (e.g., a control sample or a specimen/subject sample) is generally used to refer to a physical material that can be sequenced by a genetic sequencing machine. A genetic or genomic sequence (e.g., a control sequence) is generally used to refer to data or information characterizing the order of genetic code (e.g., the genetic sequence of single or double bases).

In one preferred variation, one or more pre-configured DNA control sequence(s) from the system and method can be used in generating enhanced spike-ins that can facilitate automated detection and analysis processes within a genomic analysis platform. In some variations, the DNA control sequence is encoded with error correcting features for enhanced detection and analysis at various stages of DNA assay handling and processing.

In particular, robust next-generation sequencing (NGS) metagenomic assays can benefit from defined detection limits and process traceability from sample collection to bioinformatic analysis. The genetic control samples (e.g., the enhanced spike-ins) of the system and method can serve as qualitative controls used to barcode and track samples during genomic sample processing. The genetic control samples may additionally provide absolute concentration data to address these challenges.

In one particular use case, the system and method are used in connection with a genomic-based computing platform, application, or service. Such a genomic computing platform may be used for analyzing collected genomic data. The system and method when integrated into the operation of the genomic computing platform can automate spike-in detection and tracking. Furthermore, after detection and tracking, the system and method may use the metadata information communicated through detection of the control sample, to facilitate process workflow automation: generating reports, generating notifications or alerts to detected process events and conditions (e.g., reporting false positives, false negatives, cross contamination, etc.), machine/process detection, concentration estimation, automated analysis based on metadata information, and/or other processes.

As one example, a microbial genomic computing platform designed to help applied microbiologists rapidly assess their samples may apply the system and method for automated sample identification and process control. Then, an experimenter can use a set of DNA control sequence samples as a form of enhanced spike-ins during experiment administration to automate labeling and tracking of samples within the microbial genomics computing platform. The system and method may automate internal controls of an experiment and/or provide other functionality within a software system.

Additionally or alternatively, the system and method may be used in augmenting the operation of a device such as a sequencing device or system. In one variation, DNA control sequences may be used in specimen genetic samples to augment operation of a sequencing device. For example, a sequencing device may be preconfigured to change its operating mode based on the detected DNA control sequence and the detected information. While the system and method may be used for post sequencing analysis, the system and method may additionally be integrated to work in substantially real-time so that conditions can be detected and responded to with appropriate actions during a sequencing process.

The system and method can be applied to variety of genomic or bioinformatics processing systems and/or computing solutions. The system and method may be applied to a variety of DNA and/or RNA analysis use cases, wherein more precise and automated control generation may both speed up and improve the accuracy of experimental results and improve process control. The system and method is particularly applicable to next generation, high throughput, DNA sequencing. The efficient generation of DNA control sequences may be particularly useful in a field wherein large quantities of samples are analyzed depending on a large set of distinguishable spike-ins usable for process management. The system and method may alternatively be applied within analysis equipment, sequencing equipment, or any other suitable system or process.

As one potential benefit, the system and method can automate one or more processes related to managing and analyzing results of genetic sequencing. The system and method may provide automatic detection and analysis of one or more types of control samples, which can be used for automatically reporting on positive controls, negative controls, and/or internal controls.

As one potential benefit, the system and method enable traceability throughout handling of a genetic/genomic sample. The genetic control sequences of the system and method can be encoded with or configured to map to one or more pieces of metadata such as a lot identifier, a unique identifier/tag, and/or other information. The control sequences can be selected and/or generated to avoid interference with genomic samples of interest in the genomic assay (e.g., a DNA assay or RNA assay). When such control sequences are used as spike-ins, genomic assays may be better tracked.

As a related potential benefit, the system and method enable an enhanced spike-in control sample that is auto-descriptive, wherein the metadata or information can be obtained directly through sequencing the control sample and decoding the encoded information of the genomic sequence. Furthermore, the enhanced spike-in control sample encodes the metadata information using an error correcting code such as a repetition code like a Hamming code that can improve ability to recover the metadata despite various forms of genetic base substitutions, insertions, and deletions during sequencing or test processing.

As another potential benefit, the auto-descriptive qualities of the control samples can enable the control samples to open a channel for adoption of more open testing protocols where the control samples can be used as physical tags in tracking information and possibly communicating instructions. The embedded information or metadata may follow a defined protocol for the interpretation and response to the information. In one implementation, a set of control samples may be generated with the embedded information following a protocol for specifying information and purpose of the control sample. In some variations, a set of selected control samples may be used in combination during processing of a test sample. For example, different stages of test sample handling can be tracked through uniquely self-identifying identifiers embedded in control samples added during processing.

As a related benefit, automated detection and analysis of control sequences by a computing platform may be used to improve experimental process control. Genomic data processed by the computing platform could be automatically processed for control sequence detection and analysis in enabling various process control features like automated barcode/identifier entry for a sample of genomic data, cross-contamination detection, machine/process detection, concentration estimation, and/or other features.

As another potential benefit, the system and method can facilitate efficient creation and implementation of such control sequences and a resulting control sample that can be used as an enhanced spike-in. In some variations, the system and method may facilitate the automated generation of control sequences thereby allowing a large number of individually tracked samples by the enhanced spike-ins of the system and method. For example, a user may be permitted to supply a set of metadata to be embedded in a control sample, and the system and method can automate the transformation of such information into a format used in the generation of a control sample. The control sample may be generated and synthesized directly through the system and method. The control sample may alternatively be specified and the control sequence communicated to an outside synthesis system. This is particularly beneficial to next generation DNA sequencing, enabling quicker control analysis of large quantities of samples.

Another potential benefit of the system and method comes from the automated generation of the control sequence. Automated generation of batches of unique control sequences and their use with DNA assays may reduce the amount of human error.

Unique batches of control sequences may also lead to enabling detection of cross contamination. By identifying a control sequence in a sample or well that did not initially contain the control sequence, cross contamination may be significantly reduced.

As another potential benefit, the system and method may use prescribed concentration and/or quantities of control samples such that the control samples can be used in test sample quantification. Precise knowledge of production outputs may additionally be beneficial to machine calibration. In contrast to “homemade” batches of spike-ins, precise production quantities of the DNA control sequence may enable precise measurements of errors and give better understanding of the biases in analytical machines and devices.

The system and method may have a variety of additionally or alternative benefits.

### 2. System

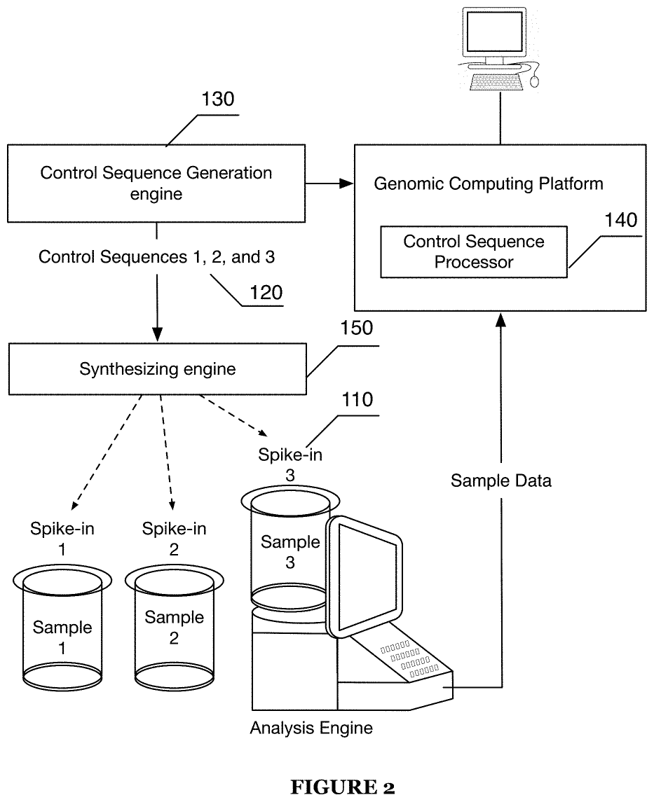

As shown in FIG. 1, a system of a preferred embodiment for application of an enhanced spike-in with a genomic computing platform can include a genetic control sample 110; and/or a control sequence processor 140 configured to analyze genetic data and actively respond to detection of the control sequence. In a preferred variation of the system used to create and/or use an enhanced spike-in, the genetic control sample 110 may be comprised of a unique genetic control sequence 120 that encodes data information. The system may additionally include a control sequence generation engine 130. The control sequence generation engine 130 and/or the control sequence processor 140 can be integrated into a genomic computing platform used for digital management and administration of genomic analysis, data management, and/or lab management system. In some variations, the genomic computing platform may be integrated with a laboratory information management system (LIMS) or similar system. In some variations, the system may additionally include an optional synthesizing system 150, whereby the synthesizing system 150 synthesizes one or a batch of the genetic control samples 110 according to specified generic control sequences 120 as shown in FIG. 2. The system functions to enable an automated genetic sequencing analysis system by introducing an automated system for control sample synthesis and analysis.

The genetic control sample no of a preferred embodiment functions as the control sample detectable by a genomic computing system. A preferred variety of genetic control sample no is a synthetic control sample no with encoded information that can be used as a spike-in for genetic specimen testing. A synthetic control sample no with encoded information can be used as an internal control. Alternatively, the embedded information may be used for alternative barcoding or other purposes. Preferably, such a genetic control sample no can be used as an enhanced spike-in for a DNA assay. A genetic control sample no is preferably a genetic sample created to have the genetic control sequence 120. The genetic control sample 110 will generally be made as a synthetic oligonucleotide.

The system may additionally or alternatively involve the use of organic control samples no and/or other types of synthetic control samples no (which may not include embedded/encoded information). Select control samples no may be registered within the system such that detection of their associated genetic sequence can trigger appropriate control processing. In one preferred variation, an organic control sample 110 can be registered and identified as a pre-configured positive control sample. In the positive control case, a control sample 110 may be made as a mixture of cells, genomic DNA (gDNA), or other biological material from known organisms. Detection of such a positive control sample can trigger automatic positive control analysis. Additionally or alternatively, a control sample no may be used as an internal control in some cases without embedded or encoded information. An organic or synthetic sequence can be registered such that associated genetic samples can be used as internal controls used in identifying and tracking a genetic assay. In one preferred variation, a synthetic control sample no may include a genetic control sequence 120 and be generated by an alternative control sequence generation engine 130 such that the sequence is selected to avoid conflicts with expected specimens.

The genetic control sequence 120 functions as a genomic sequence particularly configured for detection and use in connection with the analysis engine and/or other suitable analysis processes. The genetic control sequence 120 preferably specifies a DNA sequence or other suitable type of genetic sequence. The genetic control sequence 120 when synthesized or otherwise physically produced as the genetic control sample 110 can be usable as an enhanced spike-in. Genetic control sequences 120 are preferably DNA sequences produced or output by the control sequence generation engine 130.

The genetic control sequence 120 preferably includes encoded information. The encoded information can make the genetic control sequence 120 and the resulting genetic control sample automatically self-descriptive. Being self-descriptive can mean that with knowledge of the decoding process others may be enabled to decode and extract the embedded information. This may be done without prior knowledge of the exact genetic control sample no.

The genetic control sequence 120 preferably includes information encoded through an error-correcting or other code such as a repetition code or more specifically a Hamming code. The information can include a set of metadata. The information can serve as a detectable identifier (i.e., a “genomic barcode”) and used in identifying metadata of a sample. In another variation, the information can be used in referencing externally stored metadata or data records stored in a database system.

The information preferably includes an identifier (e.g., a unique identifier). The identifier may be a globally unique identifier. Alternatively, the identifier could be locally unique. For example, one experimental kit that includes a set of enhanced spike-ins (each associated with one genetic control sequence 120) may each have a unique identifier. This identifier may however, be repeated across distinct kits.

The information in one variation may additionally include a lot number, which functions as a group identifier. Lot numbers may be particularly useful for tracking production quality of inputs in a quality controlled experimental/wet setting.

The information may additionally or alternatively include any suitable information. The information may include one or more properties which can include metadata information such as sample sequence production location, production date, instrument that created the sample sequence, spike-in sample concentration or quantity information, a spike-in lot or group identifier, an experiment identifier, a tracking number for the sample sequence, an identifier of an associated genetic test kit, an identifier of a manufacturer, and/or other suitable metadata.

Detection of all or parts of a genetic control sequence 120 during genomic analysis can be used to automatically trace samples, measure cross contamination, estimate genomic concentrations, access spike-in metadata, and/or provide other features.

There is preferably a set of different genetic control sequences 120 used within the system. A set of genetic control sequences 120 can be synthesized and offered as a kit usable by an experimenter for tracking multiple DNA assays.

In some variations, the system may include a sample container prepared with a genetic control sample 110. The sample container may contain a known quantity or concentration of the genetic control sample no. Furthermore, the information stored within the genetic control sequence 120 of the genetic control sample 110 can be stored in association with sample container information. In one variation, a physical label on the sample container can be associated through a datastore with the information encoded in the genetic control sequence 120.

The control sequence generation engine 130 of a preferred embodiment functions to produce, generate, or otherwise output the specifications of one or more genetic control sequences 120. The control sequence generation engine 130 is preferably an application, script, or computer automated process configured to generate and/or validate genetic control sequences 120. The control sequence generation engine is preferably operable on a computer, processor, circuit, or other suitable computing system implementation. The control sequence generation engine 130 may be incorporated into a genomic computing platform. For example, the control sequence generation engine 130 may be operable on a server that can generate genetic control sequences 120 for a requesting computer client device.

The genetic control sequences 120 produced by the control sequence generation engine 130 may be formatted as sequence data that can be communicated or otherwise used to direct the synthesis engine or an outside lab. In one variation, the control sequence generation engine 130 can be directly integrated with a synthesis engine 150 such that a machine or system may produce enhanced spike-in samples directly controlled by the generation engine.

In one preferred variation, the control sequence generation engine 130 generates synthetic oligo sequences used to define a genetic control sequence 120. The generated sequence preferably satisfies a set of control conditions, which functions to verify suitability for detection, feasibility of synthesis, avoidance of experiment interference, and/or other features. In one preferred implementation, the control sequence generation engine 130 is configured, when processing a genetic control sequence 120, to: verify sequence uniqueness; verify the sequence is free of homopolymers; the sequence contains unique k-mers for a specified k; the sequences doesn't contain common adapters or other artificial sequences common in one or more sources (e.g., the NGS libraries); and satisfies synthesis feasibility conditions.

In verifying uniqueness, the genetic control sequence 120 is preferably verified to not be homologous to known reference genomes. The control sequence generation engine 130 may compare the sequence against third-party databases (e.g., the nr database compiled by the National Center for Biotechnology Information) or internal databases. Comparison may use Basic Local Alignment Search Tool (BLAST) or any suitable tool. Additionally, the uniqueness may perform a specified k-mer comparison (e.g., a 31-mer). Accordingly, the control sequence 120 output from the control sequence generation engine 130 is preferably a unique sequence with unique sub-sequences (e.g., unique k-mers)

In monitoring for homopolymers, sequence length may have any suitable threshold such as n≤3. For example, sequences like “AAAA” would not be permitted within a genetic control sequence 120. Accordingly, the control sequence 120 output from the control sequence generation engine 130 preferably does not contain a homopolymer above a threshold (e.g., a threshold of 3).

The synthesis feasibility conditions may include verification of a suitable tertiary structure and/or other control conditions. These control conditions may be used in any suitable combination and additional or alternative checks may similarly be used.

In one variation, the control sequence generation engine 130 can encode information into the genetic control sequence 120. Alphanumeric information or any suitable type of data payload can preferably be encoded into at least a portion of the genetic control sequence 120 that satisfies the control conditions. Preferably, an alphanumeric code is encoded into a genomic representation. The encoding preferably utilizes error correction. An error-correcting encoding may make enhanced spike-in detection robust to DNA base substitutions, insertions, and deletions. Such errors can occur during manufacturing or as part of sequencing.

The control sequence generation engine 130 preferably includes machine instructions interpretable by a processor, circuit, or computer system and configured to: generate a random template binary sequence, transform the embedded information into a base four data message, encode the base four data message with an error-correcting or other code such as a repetition code, and apply a bitwise XOR operation of the template binary sequence against the encoded sequence. More generally, the control sequence generation engine 130 encodes a message, which can be represented in a binary format, and then applies a transformation function that introduces a degree of randomness or a higher level of entropy than the encoding may have generated. An XOR operation is one possible option, but an entropy enhancing transformation operator could alternatively be a hash operator, a cryptographic operator, a custom function defined by a set of transformation rules, and/or any suitable type of transformation operator. A preferred property of the transformation operator is that it is reversible. Herein references to the XOR operator is used as a convenience for describing one particular variation, but one skilled in the art could appreciate that the transformation function may be a variety of other operators.

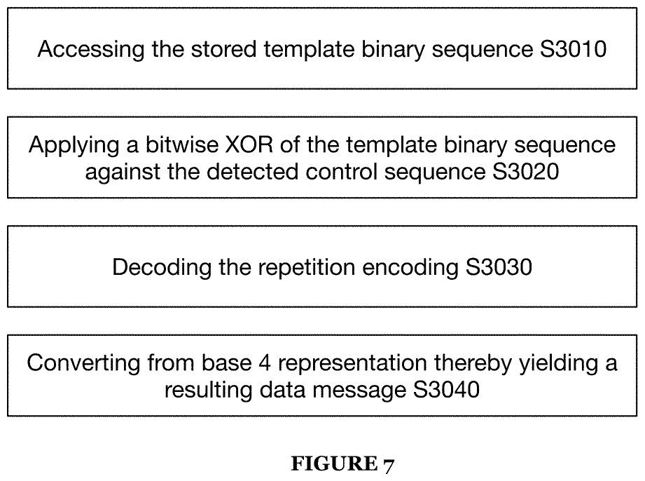

The machine instructions may additionally include configuration to validating a set of control conditions of a resulting control sequence. Validation is generally configured for execution after applying the bitwise XOR operation. If a control condition is not satisfied, the control sequence generation engine 130 is configured to generate an updated control sequence by encoding the embedded information using a different random template binary sequence as shown in FIG. 8.

In a preferred variation of execution of the configured instructions of the control sequence generation engine 130, a random template binary sequence is initially generated. Then data information such as a sequence descriptor, a barcode identifier, and manufacturing lot information, is inserted into the template binary sequence. As an example, the information data can be transformed into base four, and then encoded with an n=3 repetition code (i.e., a repetition code of length 3), where each 2-bit pattern (0b00->A, 0b01->C , 0b10->G , 0b11->T) is repeated three times. This may be implemented as a Hamming(3, 1) code. The resulting base four pattern is then bitwise XORed against the template binary sequence. This can function to facilitate a sufficiently random nucleotide string. The template binary sequence is preferably of identical length to the base four pattern. Decoding (used later during detection of the control sequence) may then be as simple as applying a bitwise XOR of the template binary sequence against the detected sequence and then decoding the Hamming(3, 1) code. Other alternative approaches such as a DNA fountain approach may alternatively be used.

The resulting sequence is then validated against the control conditions. If the genetic control sequence 120 does not satisfy the set control conditions, the initial random template binary sequence may be perturbed or the process otherwise altered until a suitable resulting sequence usable as a genetic control sequence 120 is achieved.

Finally, the control sequence generation engine 130 can establish unique primers, which may allow for detecting the sequence as well as potentially facilitate Sanger sequencing for orthogonal confirmation of the sequence. The information is preferably embedded in only a portion of a resulting control sample.

One exemplary output of the control sequence generation engine 130 using the above process may include a tag region, wherein the tag region includes DNA encoded data, and a body region that comprises of the majority of the control sequence as shown in FIG, 3. Other data-based features can additionally be layered or integrated into the encoding process. For example, cryptographic encoding may be used in securing the encoded data.

In another variation, the control sequence generation engine 130 can generate a genetic control sequence 120 through an alternative approach and then store a data record that is mapped to the genetic control sequence 120. In one approach, random or pseudo-random sequences may be iteratively generated and compared to the control conditions. Sequences that satisfy the control conditions can be saved as potential genetic control sequences 120. The potential genetic control sequences 120 may then be associated or mapped to a data record when they are selected for use. For example, the sequence descriptor, barcode identifier, and manufacturing lot information can be stored in the data record. Upon detection of the genetic control sequence 120 in a sample, the data record can be accessed.

In one implementation, the control sequence generation engine 130 may be integrated with an application (e.g., an online website or locally running application or script). The application preferably includes a user input interface so that a user can supply desired metadata information. Multiple property values may be supplied. After setting the information, the control sequence generation engine 130 can verify a suitable genetic control sequence 120 can be created and then a control sample order interface can be used to facilitate ordering of an associated genetic control sample no. Once ordered, an order request specifying the genetic control sequence 120 can be communicated to an internal or external synthesizing system 150. Alternatively, the control sample 110 may be produced directly or through other suitable forms of specifying the genetic control sequence 120 to a system for genetic sample production.

The synthesizing system 150 of a preferred embodiment functions to synthesize or otherwise manufacture one or more samples of genetic control sequences 120. Preferably, a set of different genetic control sequences 120 may be produced as a kit that can be provided to experimenters for use as a spike-in. The amount of the genetic control sequence 120 within each batch is preferably known and controlled. The synthesizing system 150 preferably creates each batch of genetic control sequence 120 to be distinct and easily distinguishable (through the encoded data model), by the analysis engine, from other batches of genetic control sequence 120.

A genomic computing platform functions to apply the detection of genetic control sequences 120 in the management of genomic analysis. In the broader context, the genomic computing platform is preferably used to facilitate analysis and processing of genomic information. In an exemplary instantiation, users or DNA sequencing machines may communicate genomic sample data to the computing platform, which will generate a report characterizing the biological classifications of sequence fragments identified in the genomic sample data. The computing platform may additionally or alternatively provide other suitable functionality.

The genomic computing platform can be any suitable form of computer executable service. The genomic computing platform is preferably a system comprised of a set of computers, data storage systems, databases, and/or other suitable processing and computer devices. The genomic computing platform is preferably cloud-implemented hosted on remote servers and connects with external client devices accessing the genomic computing platform over a network. In a preferred implementation, the genomic computing platform is a platform that enables multiple distinct accounts to individually use the functionality of the genomic computing platform. Such an implementation may operate as a cloud-hosted service where different users create accounts to process their respective genomic data. In another implementation, the genomic computing platform may be provided as an application that can execute on the computing resources managed by the user or any suitable entity. The genomic computing platform will preferably include a user interface component in presenting information related to genomic information. The genomic computing platform will additionally include other suitable components.

The genomic computing platform preferably includes a control sequence processor 140, which functions to detect a genetic control sequence 120 and access associated information data. Detection of the genetic control sequence 120 preferably comes through the normal biological classification/search process of the computing platform. The set of possible genetic control sequences 120 are preferably registered and stored within genetic database of the genomic computing platform or other identification system such that a genetic control sequence 120 can be detected in a parallel process to other biological sequence classifications. In another variation, the generated genetic control sequences 120 of the control sequence generator engine are stored in a control sequence database. The computing platform preferably customizes handling of the detected control sequence fragments. After initial identification, the associated information is extracted.

Preferably, the control sequence processor 140 preferably applies a decoding process to access the associated data. The decoding process is preferably an inverse process to the encoding process.

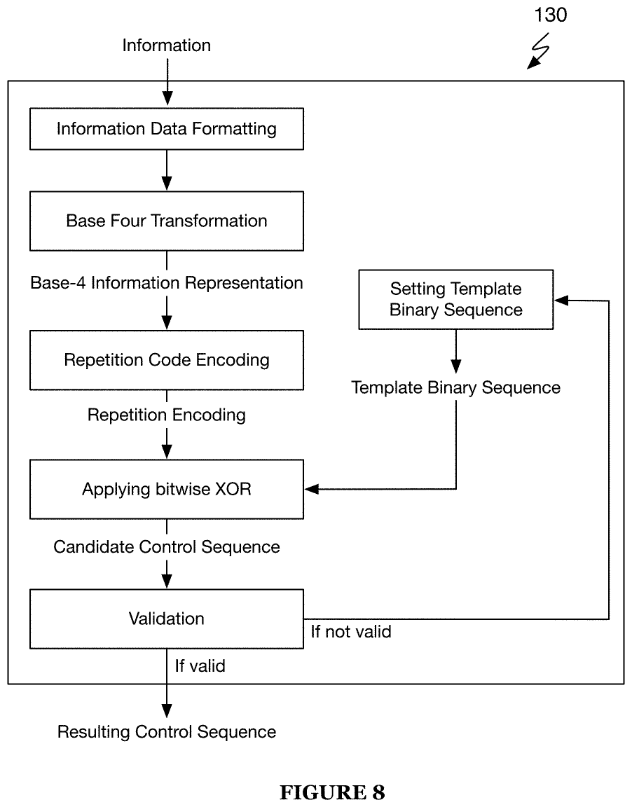

The control sequence processor 140 preferably includes machine instructions interpretable by a processor or computer system and configured to access the stored template binary sequence (i.e., the key); apply a bitwise XOR of the template binary sequence against the detected control sequence; decode the repetition encoding; and convert from base four representation thereby yielding a resulting data message as shown in FIG. 9.

In the variation of the genetic control sequence 120 and/or the encoded information being mapped to a data record, a classification identifier (applied to the genetic control sequence 120 and detected when performing classification) and/or the metadata information may be used to retrieve a corresponding data record.

Additionally or alternatively, the control sequence processor 140 may be used in systems other than a genomic computing platform. For example, sequencing equipment may use a similar control sequence processor to detect and respond to sampling of genetic control sequences 120.

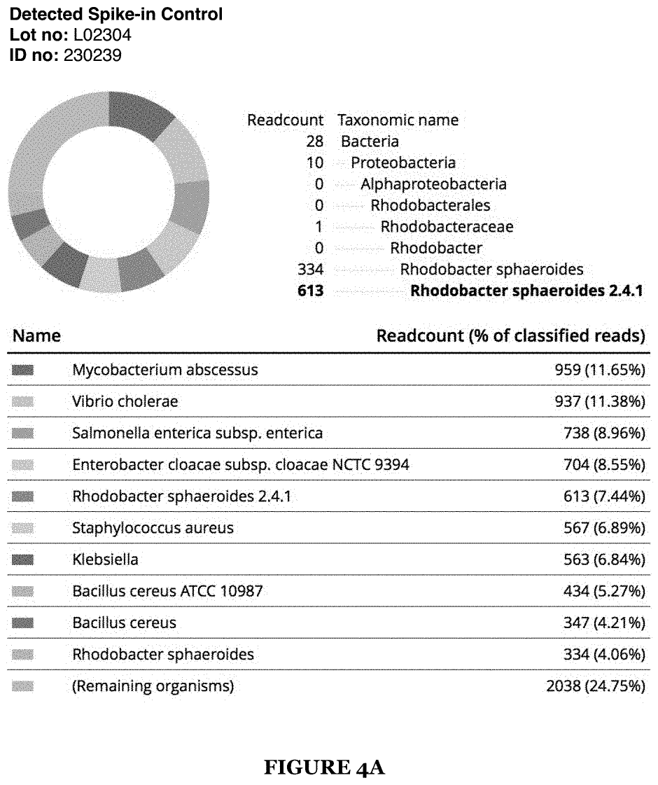

Preferably, the computing platform may respond to the detection of a genetic control sequence 120 in a variety of modes. In one mode variation, detection of a genetic control sequence 120 is used to access an identifier, which may be associated with information such as an experiment number, a particular source of a sample, or any suitable information. In the user interface, this identifier and/or the associated information can be presented within an analysis dashboard. More specifically, the detection of the genetic control sequences 120 may be removed from the genomic sample classification information and instead applied as contextual information as shown in FIG. 4A.

In a similar variation, detection of a genetic control sequence 120 in multiple different samples may be used to automatically group the associated samples and their results. This may be applied to track samples along different stages of a process. For example, multiple genomic samples collected at different stages of handling can be used to form a timeline of a sample. This may alternatively be used to track similar iterations of repeated processes. For example, the same enhanced spike-in may be used to repeat multiple iterations of testing the same sample. Since they all use the same spike-in they can be grouped together automatically by the computing platform. In such a variation, a group analysis report may be generated in response to identifying shared genetic control sequences 120.

In another mode variation, detection of multiple genetic control sequences 120 may be used to flag potential cross contamination. Detection of two or more genetic control sequences 120 in some instances may be a sign that two or more samples with enhanced spike-ins came into contact during handling of a sample. An alert may be communicated by the computing platform to indicate such potential issue as shown in FIG. 4B.

In another mode variation, the computing platform may be preconfigured with expected quantities and/or concentrations of the spike-in used in a sample. Through this, relative counts of sequence detection between the genetic control sequence 120 and other sequence fragments can be used to estimate quantities and concentrations of other biological or other forms of classifications. For example, if an enhanced spike-in has a million copies per microliter and then the computing platform can compare the number of spike-in reads to those of other specifies to estimate a concentration estimate of the detected organisms.

### 3. Method

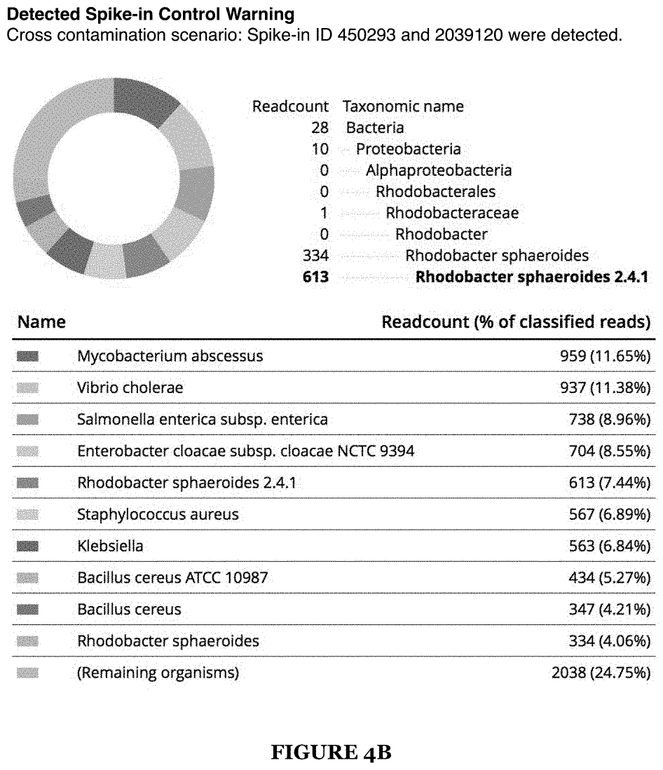

As shown in FIG. 5, a method for application of an enhanced spike-in with a genomic computing platform includes specifying a control sequence S10, applying use of a control sample that is generated from the control sequence S200, detecting the control sequence in sequencing results for a subject sample S300, and augmenting sample management within the computing system according to the detected control sequence S400.

In one preferred alternative using synthetic control samples with embedded information, the method preferably employs the use of error-correcting codes and other supplemental processes to transform information (e.g., barcoding information, sample metadata, etc.) into a genomic control sequence. A control sample can be generated from the genomic control sequence and then used as an enhanced spike-in when processing a subject genomic sample (i.e., a specimen). When sequencing the subject genomic sample, presence of the genomic control sequence can be detected and the embedded information extracted. The information can then be used in augmenting a computing system, augmenting genomic sequencing/testing equipment, and/or triggering any suitable digital response.

In one variation, the method may make use of one or more synthetic or organic control sequences such that use of a corresponding synthetic or organic sample exhibiting the control sequence can trigger the computing system to augment sample management in pre-defined manner. In one variation, an organic control sequence may be designated as a positive control (and possibly selected to not be commonly found in normal specimen samples) such that detecting a genomic sample exhibiting the organic control sequence triggers the computing system to treat that sample as a positive control.

In some variations, the method may include specifying multiple distinct control sequences such that sample management may be dynamically adjusted differently depending on which control sequences are detected. In this way, the method can provide a method through which internal controls can be used to barcode one subset of genomic samples, another positive controls can be used to automatically evaluate a positive control genomic sample. Additionally, negative controls may be detected in a similar manner. Though in some variations, negative controls may be detected through low quantities of sample data and then automatically treated as a negative control.

In method variations with information embedded controls, Blocks S100, S200, S300, and S400 generally characterizes application of the enhanced spike-in from creation of an enhanced spike-in through to detecting a enhanced spike-in and augmenting a computing system based on the information obtained during detection. Variations of the method may, however, incorporate any suitable combination and permutation of the processes S100, S200, S300, S400 and/or other various processes.

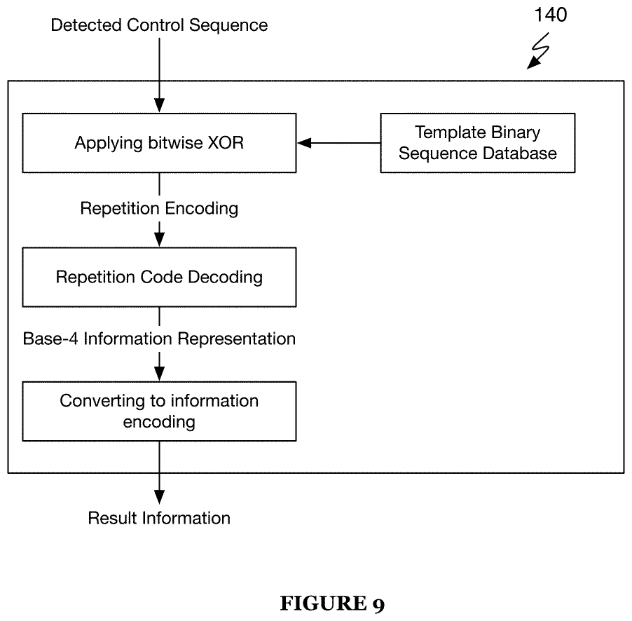

For one variation, an alternative method variation for information embedded may be applied to the generation and creation of a control sample to be used as an enhanced spike-in. For example, supplied information may be processed and transformed into a genomic control sequence that encodes the information and then a physical control sample may be synthesized from the genomic control sequence specifications. In one variation of the method, the method may include at a control sequence generation engine: encoding embedded information into a genomic control sequence S110, registering the genetic control sequence to a genetic database of the computing system S120, and obtaining a control sample based on the control sequence S130 as shown in FIG. 10.

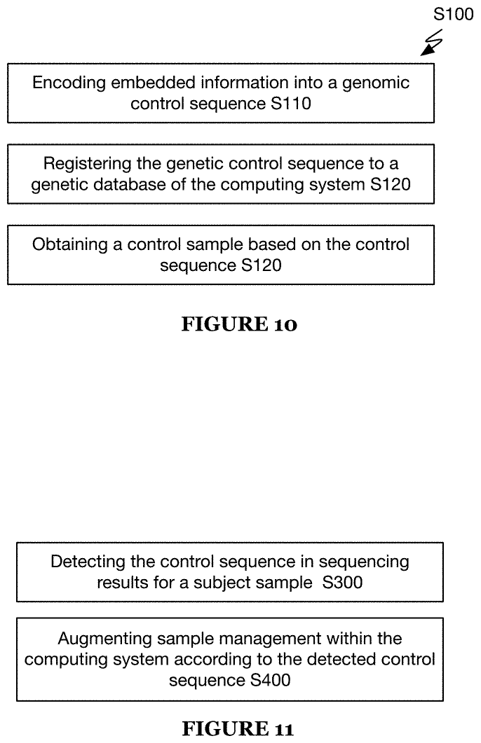

As another example, an alternative method variation information embedded controls and/or other types of controls may specifically be applied to the sequencing of a control sample and decoding of embedded metadata thereby transforming a detected presence of the control sample information that can be used to trigger some action. In the case of information embedded controls detection can be used to trigger extraction of embedded information. In the case of other types of controls, an alternative method variation can include altering operation of a computer device or system according to the metadata associated with the controls such as labeling the control sequence as a positive control sequence. In one variation of the method, the method may include at a genomic computing platform, detecting the control sequence in sequencing results for a subject sample S300, and augmenting sample management within the computing system according to the detected control sequence S400 as shown in FIG. 11

The method is preferably used in combination with a system as described above, but any suitable system may be used. The method may function in aiding and improving quantitative measurements of a DNA assay or RNA assay by providing a distinct, standardized spike-in control for each experimental sample.

In one preferred implementation of the method, the method includes encoding information into a genetic control sequence through an error-correcting code S110; at an computer database of the computing system, registering the genetic control sequence to a genetic database; in association with a genomic sample, detecting a detected instance control sequence in a collection of genetic data collected for the genomic sample S300 and decoding the control sequence thereby obtaining result information; and augmenting sample management of the genomic sample within the computing system according to the detected control sequence and the result information S400.

As yet another description of the method, the method will generally be used with a variety of control sequences and individually detecting those control sequences so as to distinguish between two control sequences with different encoded information. Accordingly, a description of the method used with at least two control sequences can include: encoding a first set of information into a first genetic control sequence through an error-correcting code S110; obtaining a first control sample based on the first genetic control sequence S120; detecting, in association with a first genomic sample, an instance of the first control sequence and decoding the first control sequence to obtain the first set of information S300; encoding a second set of information into a second genetic control sequence though the error-correcting code S110; obtaining a second control sample based on the second genetic control sequence S120; and detecting, in association with a second genomic sample, an instance of the second control sequence and decoding the second control sequence to obtain the second set of information S300. The first and second genomic samples may be the same specimen or different specimens.

Block S100, which includes specifying a control sequence, functions to establish a control sequence that can be configured for automated management by a computing system. Specifying a control sequence can include generating, identifying, selecting, or otherwise configuring a sequence such that control samples exhibiting that genetic sequence signature can be used as a trigger for dynamic processing in the computing system. Control samples can include internal controls (barcoding spike-ins) or positive controls. In some variations, negative controls may additionally be registered.

Specifying a control sequence may include generating a synthetic control sequence. The control sequence may be generated so as to encoded information that can be used in the creation of a control sample. Alternatively, the control sequence may be a synthetic sequence that can be uniquely identified while satisfying a number of control conditions. In yet another variation, the control sequence can be an organic control sequence. An organic control sample in particular may be specified as a positive control and used in automatically reporting positive control performance. Suitably unique organic control sequences may also be specified for use as an internal control. Preferably, an organic control sequence can be selected to be substantially rare for the intended sample space of intended or expected genetic analysis.

The control sample is preferably used during genomic sample handing and processing in a laboratory or test scenario as an enhanced spike-in. The control sequence is preferably specified as amino-acid sequences of different proteins or nucleotides of DNA sequences. Similarly, the system can additionally or alternatively be applied to RNA or other suitable biological sequence information. Herein, the system is primarily discussed as it applies to biological sequence information. Preferably, specifying a controls sequence includes encoding embedded information into a genomic control sequence S110. Specifying a control sequence preferably includes registering the genetic control sequence to a genetic database of the computing system S120. Specifying a control sequence may additionally include obtaining a control sample based on the control sequence S130.

As a spike-in with embedded information, the control sample is auto-descriptive in that the genetic sequence when sequenced can be transformed into a specified message. The control sequence can preferably be generated based on supplied information. Accordingly, generating the control sequence preferably includes establishing association of a control sequence with a data model. Establishing an association can include receiving specification of the data model. Specification of the data model can be supplied through a user interface and/or a programmatic interface. In one variation, a user may be able to supply data values for one or more properties when designing or specifying a custom control sequence. This can be done through a graphical user interface (e.g., a form field on a website or application), a command line interface, or through any suitable interface. In another variation, the information may be programmatically supplied such as through an application programming interface (API). A programmatic interface may enable interfacing with an outside computing system or device for specification data model information.

The data model is preferably information that serves as a set of metadata, key-value pairs, or any suitable type of descriptive data that can be communicated in the provided data-space within the control sequence. In general, the data model will be information specifying one or more parameters.

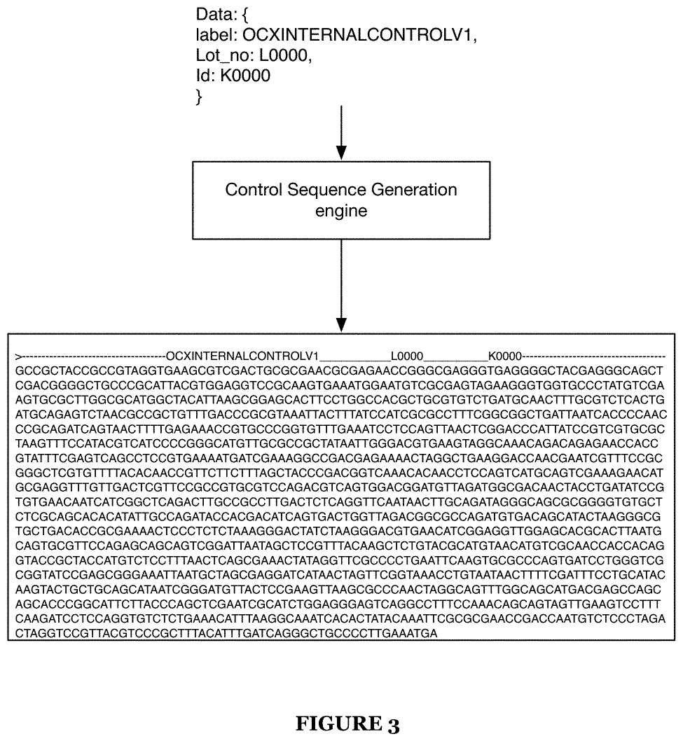

In one preferred implementation, the data model (i.e., “information”) could include various forms of metadata and/or identifying labels. For example, the data model may include information such as the sample sequence production location, production date, instrument that created the sample sequence, spike-in sample concentration or quantity information, a spike-in lot or group identifier, an experiment identifier, a tracking number for the sample sequence, an identifier of an associated genetic test kit, an identifier of a manufacturer, and/or other suitable metadata. The data model can be formatted in any suitable manner. Preferably, the data model is a data message, which can be an alphanumeric string that adheres to a prescribed format to specify various pieces of information. A set data-protocol may be defined wherein various property values may be efficiently supplied. For example, an alphanumeric label and a lot number may be two fields, which are encoded into the control sequence in a specified order and placement when encoding into a genetic sequence. As shown in the example of FIG. 3, the data message may be represented as three pieces of information with filler. Some exemplary pieces of information can include a spike-in text-based label, a lot or group identifier, and a unique identifier.

Block S110, which includes encoding embedded information into a genetic control sequence, functions to specify or generate a control sample that follows the genetic control sequence. The control sequence once defined can be materialized as a synthetic oligonucleotide through block S130 or by an outside system/entity. Upon the control sequence being synthesized into a control sample, the control sample can be used as an enhanced spike-in. The information is preferably encoded in the DNA sequence pattern as a sequence of four possible characters: adenine (A), cytosine (C), guanine (G) and thymine (T). Encoding embedded information into the genomic control sequence preferably includes encoding information into the genomic control sequence through an error correcting code. The error correcting code is preferably a repetition code and in one preferred variation is a Hamming code, though a variety of other error-correcting codes may alternatively be used. As discussed above, the information can be associated with a data model and may be supplied by a user, a computing device, and/or any suitable entity. With flexibility in the contents of the information, the control sequence and a resulting control sample may be used for a variety of applications.

Encoding embedded information into the genetic control sequence S110 will preferably include two stages of processing: encoding of information and transforming encoded information into a genomic compatible sequence. Information can be converted from its original format to a data representation such as a binary format. The binary representation may be formatted and then encoded. This can include applying an error-correcting encoding operator such as a repetition code or a Hamming code. The result of this encoding may have sequence patterns can then have one or more transformations applied to make a resulting sequence that exhibits preferred properties for synthesizing, using in actual practice, and/or sequencing.

In some variations may include a third stage of verification

A transformation function is preferably applied to introduce a degree of randomness or increase the level of entropy as compared to the initial encoding. One preferred transformation variation includes applying an XOR operator on a selected key (i.e., template binary sequence) and the encoded intermediary representation is one possible option. Applying an entropy enhancing transformation operator could alternatively include applying a hash operator, a cryptographic operator, a custom function defined by a set of transformation rules, and/or any suitable transformation operator. A preferred property of the transformation operator is that it is reversible. Herein references to the XOR operator is used as a convenience for describing one particular variation, but one skilled in the art could appreciate that the transformation function may be a variety of other operators.

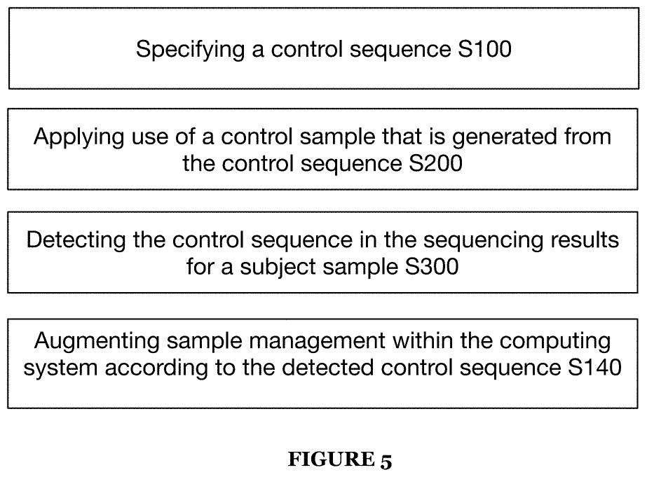

As shown in FIG. 6, one implementation of encoding information into the control sequence can include generating a template binary sequence S1110, transforming the embedded information into a base four data message S1120, encoding the base four data message with a repetition code S1130, and applying a bitwise XOR operation of the template binary sequence against the encoded sequence S1140. The result of S1140 is preferably outputting the genomic control sequence yielded from successfully encoding the embedded information, which is generally the result of the bitwise XOR operation by some conditions may first verify prior to a final result is achieved. The process for encoding information into the control sequence may additionally include after applying the bitwise XOR operation validating a set of control conditions for the resulting control sequence S1150 and if a control condition is not satisfied, generating an updated control sequence by encoding the embedded information using a different random template binary sequence as shown in FIG. 8. The encoding of information into the control sequence is preferably performed and executed at a control sequence generation engine executing on a processor, circuit, or other suitable computer system. A control sequence generator engine can establish unique primers, which may allow for detecting the sequence via PCR (Polymerase chain reaction) or other methods, as well as potentially allowing for orthogonal confirmation through Sanger sequencing. Other alternative approaches such as a DNA fountain approach may alternatively be used.

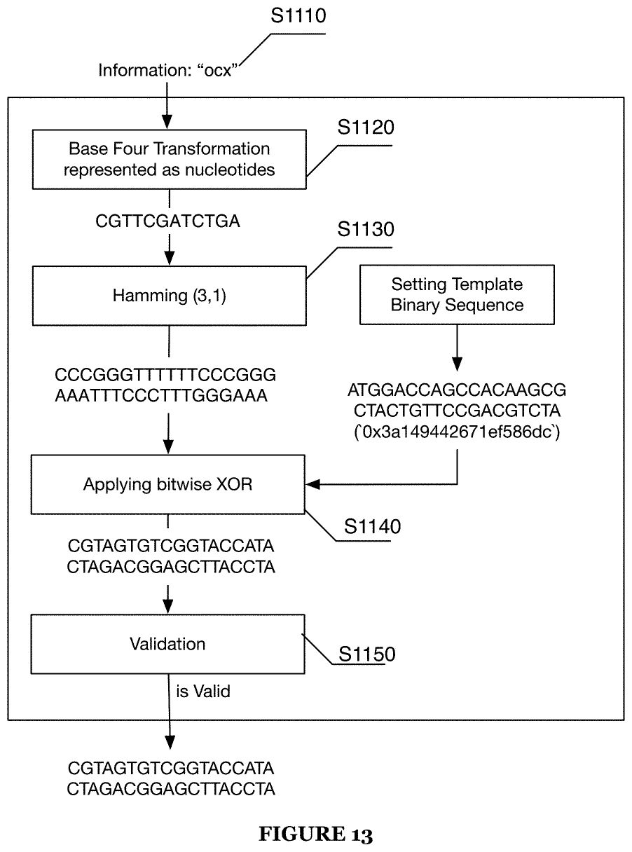

As a simplified example shown in FIG. 13, a three character message “ocx” may be simply formatted using its three characters and then converted to a base four representation that when represented as nucleotides can yield “CGTTCGATCTGA”. Applying a Hamming(3,1) transformation yields “CCCGGGTTTTTTCCCGGGAAATTTCCCCTTTGGGAAA”. A suitable key is selected such that the control conditions are satisfied. In this case, the key “ATGGACCAGCCACAAGCGCTACTGTTCCGACGTCTA” (i.e., ‘0x3a149442671ef586dc’) is used. XORing the key and the repetition encoded version yields the final result of “CGTAGTGTCGGTACCATACTAGACGGAGCTTACCTA”. A control sample may be generated with this genetic sequence and the information can preferably decoded and extracted as will be described below.

Block S1110, which includes generating a template binary sequence, functions to supply a key by which the entropy of an initial encoding of information may be made more suitable for use as a control sample. The template binary sequence can be a random or pseudo-random binary sequence. A purely random genetic sequence is often not easily manufactured as a genetic sequence as there may be random sequence patterns making it difficult for synthesizing a sample and/or for sequencing a resulting sample. Accordingly, the template binary sequence is more preferably a pseudo-random binary sequence, which may be tuned for manufacturability and/or ease of sequencing. The pseudo-random binary sequence may be tuned or adjusted through an iterative process based in part on results of block S1150. In one exemplary implementation, the template binary sequence is preferably a bit-string of identical length to the initial nucleotide string resulting from S1130. The template binary sequence can function as a key used to encode and decode. A template binary sequence is preferably identified that can be used across a number of control sequences so that they may be used as a class of control sequences where only one template binary sequence. Accordingly, the template binary sequence is preferably stored for later access and use when decoding and/or when encoding other control sequences.

Block S1120, which includes transforming the embedded information into a base four data message, functions to convert the information into a base four data message. Preferably, the binary representation of an alphanumeric string or numeric value of the information is converted into a base four data message based on the base or radix of the genetic sequence. In the case of a DNA sequence, the two digit binary sequence conversion to a DNA bases may be 0b00->“A”, 0b01->“C”, 0b10->“G”, 0b11->“T”, though other suitable mappings may be used. A computer implementation will generally maintain a binary representation and may not directly represent the data using symbolic symbols representing the genetic bases. Description of the parallels and mapping to the bases is provided herein for clarity of the description and is not intended as a set limitation as can be appreciated by one skilled in the art.

Block S1130, which includes encoding the base four data message with a repetition code, functions to use repetition as an error-correcting code when encoding the information into a genetic sequence. The repetition code can make the conveyed information more resilient to recovery despite corruption of the genetic sequence during synthesis, use of the sample, and/or sequencing. A repetition code is one preferred type of error-correcting code, but other alternative error-correcting codes may alternatively be used. In a binary repetition code, two code words can be used: all ones and all zeros with a length of n. The repetition code in one implementation preferably uses n=3, such that each 2-bit pattern (e.g., 0b00->“A”, 0b01->“C”, 0b10->“G”, 0b11->“T”) is repeated three times. This repetition code enables error correcting of (n−1)/2 such that when n=3 up to one error in any code word can be corrected. A binary repetition code of length three is used which corresponds to a (3, 1)-Hamming code. Accordingly, the repetition code is preferably a Hamming code. A (3, 1)-Hamming code has a block length of 3 and message length of 1. Other Hamming code variations such as a (7, 4)-Hamming code with a block length of 7 and message length of 4 may alternatively be used. Similarly, additional parity bits may be incorporated to enable more robust error correction and recovery from multiple instances of bit corruption.

Block S1140, which includes applying a bitwise XOR operation of the template binary sequence against the encoded sequence, functions to alter the entropy of an initial repetition encoding of the information. Block S1140 may alternatively or more generally include applying an entropy enhancing transformation operator. Applying a transformation operator may include applying: an alternative bitwise operation or set of bitwise operations, a hash operator, a cryptographic operator, a custom function defined by a set of transformation rules, and/or any suitable transformation operator. Description herein of use of an XOR operation can alternatively be performed for any suitable transformation operator. The repetition code may introduce an amount of order that can be less then ideal for genetic synthesis or sequencing. The template binary sequence from S1110 is preferably XORed with the binary sequence resulting from block S1130. Herein, XORing describes the application of the exclusive (or exclusive disjunction) as a logical operation. A bitwise XORing is performed such that for each bit of a corresponding location in the sequence is compared and a result is set as true (e.g., 0b1) when the two input bits differ (e.g., true when a first compared bit is 0b1 and a second compared bit is 0b0). After application of the bitwise XOR operation, block S1140 may include converting back to the base four data message representing the genetic sequence data (e.g., the sequence of A, C, G, and T, in the DNA sequence).

Alternative implementations may use other approaches to perturb the intermediary repetition encoded sequence. For example, a hashing function, a pre-configured sequence modifier algorithm, cryptographic encoding algorithm, or other suitable process may be used. A perturbation process preferably includes an inverse perturbation process wherein the changes to the sequence can be reversed, which may depend on having access to some external data such as a private or public key or password.

Block S1150, which includes validating a set of control conditions for the resulting control sequence functions to verify that the control sequence is appropriate for use as genetic control sample in that it is realistically producible and would not interfere with test genetic samples. If the set of control conditions is not appropriately satisfied, then the process for encoding information into the control sequence (e.g., blocks S1110, S1130, and S1140) is preferably repeated using a different template binary sequence or key. If the set of control conditions are satisfied then the resulting control sequence may be used in the generation of a control sample.

If the control conditions are not satisfied, then the block S110 may include updating the template binary sequence. The template binary sequence may be updated to selectively address issues with one or more unsatisfied control conditions. For example, if a k-mer substring of the resulting control sequence is not unique to the k-mers of a genetic database, then that portion of the template binary sequence may be selectively perturbed to satisfy that condition. Where such corrective action can be taken to the template binary sequence then full reprocessing may not be needed and the resulting control sequence and the final template binary sequence may be updated directly. The template binary sequence may alternatively be updated in a methodical sequential manner, a random manner, or in any suitable manner.

In another variation, specifying a control sequence can include producing or identifying a qualifying control sequence and associating the control sequence with a data model wherein the control sequence does not embed information. Associating here can include storing a database relationship record linking a database record of the control sequence with the metadata. Metadata can be stored in association with the control sequence potentially storing a control classification or supplemental information or data that can be used when detected. In this variation, a computing platform may store the control sequence in a manner similar to other genomic classifiers (e.g., biological classifications), but enable the detection of the particular control sequence to trigger accessing of the stored metadata. In some implementations, a random sequence, pseudo-random sequence, or otherwise non-organic and synthesized sequence can be detected which satisfies a set of control conditions. In one additional or alternative variation, no information may be explicitly encoded within the sequence, but detection of the sequence may enable a related data model to be accessed. Additionally or alternatively, other known mixture positive controls (e.g., mock microbial communities) may be associated with stored metadata such that detection within a genomic computing platform may be used to augment sample management in an appropriate manner. In such case, a control sequence can be an organic control sequence. Mapping a variety of types of controls—such as spike-in controls, positive controls, and negative controls—may be used to add automatic sample management features to an analysis system.

In the case of a positive control, a suitable positive control can be selected and upon detection of the control sample, a genomic computing platform can trigger positive control evaluation. Positive control evaluation can be used to evaluate contamination percentages, calibrate protocol and measurement bias, and/or perform other tasks. In other variations, an organic control sequence (and its associated organic control sample) may be used for internal controls. An organic internal control sequence is preferably selected with a control condition that minimizes the opportunity for the organic sample to conflict with actual measurements.

For both variations of establishing an association of a control sequence, the candidate control sequences are validated against a set of control conditions. Validating the set of control condition can check one or more conditions of the sequence. In general, the conditions verify the final control sequence does not contain undesired properties such as homopolymer characteristics which can impact feasibility for synthesis or sequencing and does not conflict with other genetic sequences of interests (e.g., naturally occurring genetic sequences). Validating control conditions of one exemplary implementation may include verifying sequence uniqueness; verifying the sequence is free of homopolymers; verifying the sequence contains unique k-mers for a specified k; verifying a lack of common adapters or artificial sequences; and/or verifying the sequence satisfies synthesis feasibility conditions. These control conditions may be used in any suitable combination and additional or alternative checks may similarly be used.

In verifying uniqueness, the DNA control sequence is preferably verified to not be homologous to known reference genomes. The control sequence and the substrings are preferably searched against one or more databases defining a genetic search space and determined to be unique or not. Verifying uniqueness preferably functions to avoid confusion of a fragment of the control sequence with a known genetic sequence fragment. For example, it is generally undesirable for a fragment of the control sequence to match a fragment of a known pathogen. The control sequence generation engine may compare the sequence against third-party databases (e.g., the nr database compiled by the National Center for Biotechnology Information) or internal databases. Comparison may use Basic Local Alignment Search Tool (BLAST) or any suitable tool. Additionally the uniqueness verification may perform a specified k-mer comparison (e.g., a k=31 31-mer). The uniqueness verification condition is preferably satisfied when there the control sequence and its substrings are fully unique in the search space.

Verifying the sequence is free of homopolymers functions to avoid complications in manufacturing and detection. A sequence of repeated bases (e.g., four or more A's) can cause issue. A homopolymer condition is preferably satisfied when no homopolymers above a threshold are detected and not satisfied when one or more is detected. In monitoring for homopolymers, sequence length may have any suitable threshold such as n=3. With a threshold of 3 no more than 3 repetitions can occur.

Verifying a lack of common adapters or artificial sequences preferably involves comparing the sequence against one or more sources like the NGS libraries. Similar to the biological uniqueness verification condition, uniqueness within artificial sequences may also avoid complications.

The synthesis feasibility conditions may include verification of a suitable tertiary structure and/or other control conditions. Secondary structure folding, antibody binding, and/or other structural or synthesis rules could similarly be incorporated.

If a resulting candidate control sequence is determined to meet the criteria of control conditions then it can be used as a control sequence. If one or more control conditions are not satisfied then the specific regions of the pseudo-random sequence may be replaced or a completely new pseudo-random sequence may be generated as described above. In one variation, generation of a qualifying candidate control sequence involves iteratively determining a candidate control until a qualifying candidate control sequence has been achieved. A template binary string can be changed during each iteration until a satisfying result is achieved. Alternatively, a pseudo-random approach to the template binary may involve controlling (e.g., locally perturbing and updating) the template binary string to address specific issues.

Upon successfully encoding information into a control sequence that satisfies a number of conditions, the method may include storing and/or registering a control sequence in a genetic data computer database of the computing system as part of block S120. Additionally, the associated information may also be stored.

Block S120, which includes registering the genetic control sequence to a genetic database of the computing system, functions to configure the control sequence (or sequences) within a computing system so that they may be later recognized and appropriately responded to. Specifying the control sequence preferably stores the sequence information within a classification system such that sequencing data may be processed and used to detect if genetic information associated with the sequence was identified in a tested sample.

Registering the control sequence may add the control sequence to the search space for a genetic sequence classification used for other biological and/or synthetic sequences. Alternatively, the database may be specific for a control sequence search space.

Registering the control sequence may include registering a control sample as an internal control (e.g., a barcoding spike-in) such that detection of the internal control sample is used in triggering internal control processing. An internal control may alternatively be used as a barcode control used as an identifier or tag for the genomic sample. An internal control can be mixed with a genomic sample or potentially reacted integrated with a genomic sample.

Registering the control sequence may alternatively include registering the control sample as a positive control such that detection of the positive control sample automatically triggers positive control reporting. In one preferred implementation, a registered positive control is an organic control sequence.

Specifying the control sequence could additionally specify when and how to detect a negative control. In some variations, specifying the control sequence may register a control sequence for an alternate purpose.

Additionally or alternatively, in the case of a control sequence with embedded information, the template binary string is preferably stored as a key such that it may be used during decoding in block S300. Preferably, a key can be used for a class of control sequence samples when a set of control sequences is created from a shared common template binary string. Alternatively, multiple template binary strings may be stored as keys for a set of two or more samples encoded using a set of two or more template binary strings.

Additionally, registering the control sequence may include storing supplementary metadata or portions of a data model associated with the control sequence. In some implementations, the encoded information may not explicitly encode the target information but instead encode one or more identifiers or data used in appropriately accessing associated data. Detection of the sequence and retrieval of the information may enable a related data model stored in the computing system to be accessed.

Block S130, which includes obtaining a control sample based on the control sequence, functions to facilitate the production of a control sample with the genetic control sequence. The obtained control sample is preferably a physical sample that exhibits a genetic signature based on the control sequence—in other words sequencing of the control sample ideally reveals the control sequence absent error or contamination. In one variation, the method may include communicating the control sequence to a genomic sequencing system to automatically direct production of the control sequence. In other words, the method may include ordering or transmitting a synthesis request that specifies the control sequence as the requested control sample. Specification of the control sequence can be communicated to an outside service, which may handle the manufacturing and production of the control sample. In one exemplary implementation of the method, a user interface or data file output preferably provides the generated control sequence, and outside actors (e.g., human users or digital systems) oversee directing production.

In some variations, the method may include physical synthesizing a control sample, and in other variations the method may include directing synthesis of the control sequence. When the method includes physical production of the control sample, the method includes synthesizing a control sample with a genetic sequence corresponding to the control sequence. The generated control sequences can be produced as spike-ins or other suitable forms of control sequence samples. A manufactured enhanced spike-in is the physical manifestation of the control sequence.

In some variations, the control sequences once generated can be logged and configured for future synthesis.

In one variation, produced control sequence or sequences may be prepared as part of a sample collection device. Accordingly, the method may include producing a sample collection device prepared with the control sample. Specimen samples can then be added to the collection device and mixed with the control sample. A sample collection device preferably includes a defined cavity or well to hold a sample. Production of a sample collection device may be used to produce genetic testing kits that are pre-configured with an enhanced spike-in. For example, a set of different control sequences may be produced in desired quantities and/or concentrations within or added to a set of different sample containers. In producing a sample collection device with an integrated control sample, a control sample may be added to at least one sample container but may alternatively be added to multiple containers in a set of containers. In one implementation, a sample container may be a well of a well tray wherein one or more wells can be prepopulated with a control sample. The same control sample may be used in multiple containers. Alternatively, different control samples with different encoded information may be used in different containers. In one variation each container has a control sample with different embedded information. In this way cross-contamination can be traced back to the container that was the source of cross-contamination.

Block S200, which includes applying use of a control sample that is generated from the control sequence, functions to use the control sample in a genetic processing workflow. As described, the control sample is preferably used as an enhanced spike-in. However, the control sample may find other applications in the laboratory and test environment. Applying use of the control sample generated from the control sequence preferably includes incorporating the control sequence with a genomic sample. The control sample is preferably used with a DNA assay or DNA sample. In some variations, block S200 is preferably facilitated by use of a human user (e.g., an experimenter) or a process control system. In some variations, block S200 may exist outside of the method, but is described here to illustrate use of a synthesized control sequence. Alternatively, application and incorporation of the control sample may be performed automatically by a testing device.

As one example, a specimen may be collected from a person. That specimen serving as the subject genetic sample is then processed so that DNA sequence data can be collected. Processing may include combining the specimen with buffers, adding beads to the sample, other sample preparation steps, processing for reading by an instrument, and then sequencing by an instrument. The genetic sample of the specimen may have the control sample added to it, which can be used to establish sample-based barcoding and tracking of handling of the specimen. This may be used for internal control, cross-contamination verification, quantity estimation, and/or other forms of process management in block S400.

In some implementations, the control sample is incorporated into a sample collection device such that in a set of sample containers each have a specified quantity of distinct control sequence-based spike-ins. In one exemplary implementation, a distinct control sample (e.g., with different incorporated data models) is incorporated into in each individual DNA assay, such that each DNA assay may be distinguished from all other DNA assays by just detecting the control sequence within that assay. The control sequence is likely detected alongside the detection and analysis of the actual genomic sample of the DNA assay. A genetic subject sample will generally be supplied externally and combined with the control sample. Alternatively, the same control sample may be used in all or several DNA assays. For example, if multiple DNA sequencing techniques and/or machines are implemented to sequence some DNA sample, the same control sample may be incorporated for all DNA assays using the same technique and/or sequencing machine. In another variation of the method, incorporating the DNA control sample may alternatively incorporate the sample sequence in a test sample that does not contain any other DNA or genetic sample. The DNA sample sequence may enable running a pure control sample to aid in machine calibration and determine the error of the specific machine. In some variations, the control sample is administered in a known quantity and/or concentration. For example, a sample collection device may have a preconfigured concentration of an enhanced spike-in (e.g., the control sequence) added to the collection device.

Block S300, which includes detecting the control sequence in the sequencing results for a subject sample, functions to respond to detection of a control sequence. More specifically, its a detected instance of the control sequence that is detected—the detected instance may differ from the expected control sequence in small ways because of possible corruption or errors in the sequence.

Detection of the control sequence preferably occurs within a computing system, wherein the computing system is configured with machine instructions for processing sequencing results. Detection of a control sequence is preferably achieved through any suitable genomic sequencing approach that may be applied to other samples. Some number of reads of segments can be collected and related back to the control sequence. As the control sequence is unique, there are preferably no read conflicts with other sequence segments. A computing platform, machine, or other suitable system is preferably preconfigured with the control sequences such that it can be detected and classified as a particular control sequence. Once detected, a data model of the control sequence is preferably accessed.

Detecting the control sequence will preferably include decoding information from a detected control sequence. The control sequence is preferably decoded through a corresponding inverse process to the encoding process. The application of the template binary mask is preferably reversed and the repetition code decoded and error-corrected to reveal a data message. The data message will preferably communicate the originally encoded information assuming non-recoverable errors were encountered.

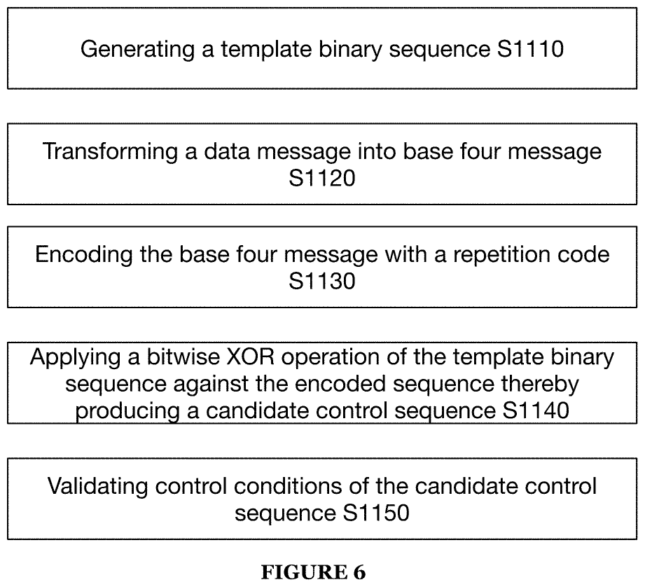

As shown in FIG. 7, one implementation of decoding information from a detected control sequence can include accessing the stored template binary sequence (i.e., the key) S3010; applying the bitwise XOR of the template binary sequence against the detected control sequence S3020; decoding the repetition encoding S3030; and converting from base four representation thereby yielding a resulting data message S3040.

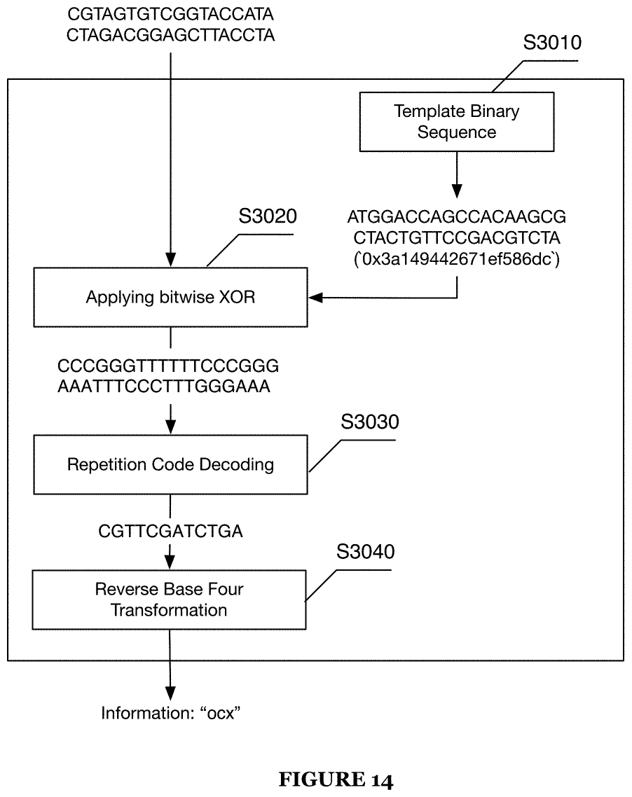

Continuing the simplified example of FIG. 13 for its respective decoding process shown in FIG. 14, sequencing data may yield the result of “CGTAGTGTCGGTACCATACTAGACGGAGCTTACCTA”. In some cases this may include some number of errors when compared to the expected sequence. The template key may be accessed. In some cases a single key is used, and in others an associated key must be selected or found. After applying the key through an XOR operation, the result is “CCCGGGTTTTTTCCCGGGAAATTTCCCTTTGGGAAA”. When error corrected, the base four representation of “CGTTCGATCTGA” can be converted to the character representation of “ocx”.

Block S3010, which includes accessing the stored template binary sequence, functions to access the sequence string used as a key when XORing. The template binary sequence is preferably stored previously from an initial generation process. In one variation, a single template binary sequence may be used across a class of possible control samples and so accessing the stored template binary sequence can access the one template binary sequence stored in memory. Alternatively, multiple template binary strings may be used across a set of possible control samples. In one instance, detection of the control sample based on its unique properties can be used to select the corresponding template binary sequence. Alternatively, a direct mapping may not be known in which case, the decoding process may be iteratively performed by iterating over a set of possible template binary sequences and evaluating the results to determine which iteration and result is likely the correct result. In some variations, a portion of the encoded sequence may serve as some checksum to verify if the correct template binary sequence is selected.

Block S3020, which includes applying the bitwise XOR of the template binary sequence against the detected control sequence, functions to inverse the XOR operation of Block S1140. As with Block S1140, Block S3020 may alternatively or more generally include applying a reverse transformation operator. Depending on the operator applied in Block S1140, the operation could be the same, an inverse operation, or any suitable operation to transform into an encoded version. Applying a reverse transformation operator may include applying: an alternative bitwise operation or set of bitwise operations, a hash operator, a cryptographic operator, a custom function defined by a set of transformation rules, and/or any suitable transformation operator. Description of XOR operations may alternatively be performed with any suitable type of transformation. Applying the XOR is preferably substantially similar to the process of S1140. The sequence is preferably converted to a binary representation using a specified mapping such as the one shown herein. The output of applying the bitwise XOR when performed on the detected control sequence preferably undoes the initial XOR operation from S110 such that the output is ideally the repetition encoded representation of the data message.

Block S3030, which includes decoding the repetition encoding, functions to obtain extracted information by interpreting the sequence through a decoding process. Decoding the repetition encoding preferably includes decoding the repetition code or more specifically the Hamming(3,1) code described above. When decoding a repetition code, a majority decision for each code word is preferably made. Accordingly, decoding the repetition encoding can include for a set of three bits in a code word, interpreting the single-error corrected state as the state of the majority of the bits (the state of at least two of the bits in the three length code word). As mentioned, the use of a 3 length repetition code is robust against corruption of one bit in a code word. Bits can be corrupted during synthesis, in sequencing the control sample, and/or at other stages in genetic processing of a sample. The output of S303 is preferably the original value unless an unrecoverable number of errors were encountered

Block S3040, which includes converting from base four representation thereby yielding a resulting data message, functions to translate the resulting sequence to an appropriate information representation. When all information is represented in character-encoding then the sequence of bits is converted to a character representation (e.g., an alphanumeric representation). In some variations, an information formatting protocol may specify different spaces for use of different types of information. These different blocks of information may be selected and individually converted according to their associated representations. For example, one block of data may be reserved for an alphanumeric label while a separate block may be reserved for an integer value identifier.

In some variations, the decoded information may be used in accessing stored data according to the decoded information. For example, the decoded information may provide a data model identifier that can be used to access more rich data stored in a database associated with the data model identifier.

In one implementation, a sequencing process executable on a computing platform will automatically search for and identify a control sample evident in a collection of sequencing data. The sequencing process can additionally automatically decode contained information.

Execution of block S300 may be independent of execution of the other blocks of the method. In some method variations, S300, and optionally in combination with S400, may be implemented in isolation from S100 and S200. For example, the specification of the control sample may be supplied elsewhere and the incorporation of the control sample performed by outside systems or entities. Blocks S300 and S400 when performed in combination can dynamically respond and detect control samples when preconfigured with some data representation and decoding processes to detect a control sample and then extract information from the detected control sequence.

Block S400, which includes augmenting sample management within the computing system according to the detected control sequence, functions to take some action in response to detection and decoding of a control sequence found in sequencing results. Within a genomic computing platform, the action is preferably to alter data management. One or more actions can be taken. Augmenting sample management is preferably performed within a genomic data management system implemented through a computer system that includes at least a processor and a data storage system (e.g., a database). Augmenting sample management can be used in automating sample data identification and organization, generating alerts or notifications to automatically detected conditions, and/or estimating quantities.

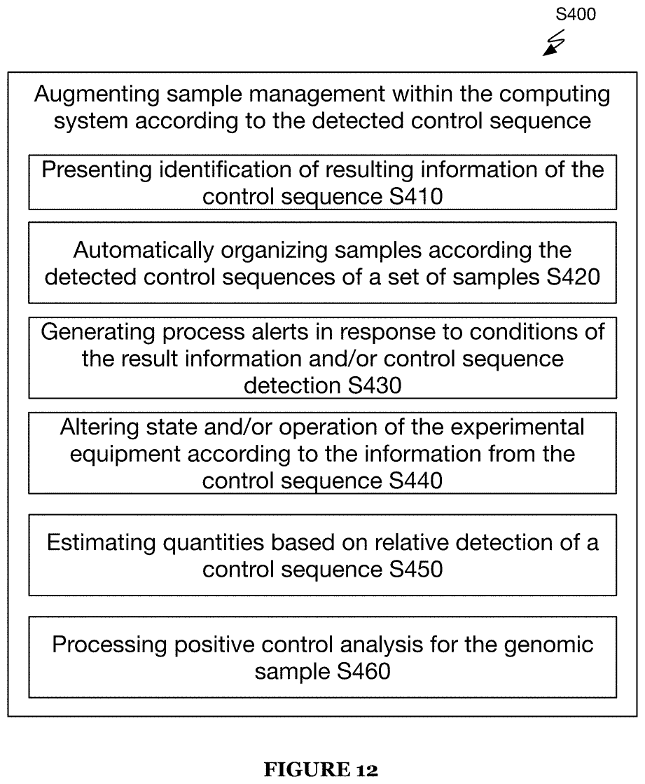

Variations for augmenting sample management, as shown in FIG. 12, may include processes such as presenting identification of resulting information of the control sequence S410, automatically organizing samples according the detected control sequences of a set of samples S420, generating process alerts in response to conditions of the result information and/or control sequence detection S430, altering state and/or operation of the experimental equipment according to the information from the control sequence S440, estimating concentration or quantity of detected sequences based on relative detection of a control sequence S450, and processing positive control analysis for the genomic sample S460. Any suitable process for augmenting sample management may alternatively be used.

As one exemplary action, augmenting sample management can include presenting identification of resulting information of the control sequence S410. Furthermore detection of the result information may trigger setting a data association of the genetic sample's (e.g., the specimen) genetic sequence data with the result information. A data association is preferably a data record stored in a database. Information from the control sample in this variation can function as a link to provide internal controls for tracking different specimen samples. As discussed, various forms of metadata may be part of the control sequence. These may be presented within the user interface when viewing. Additionally, the results of the sample may omit presenting the reads of the control sequence. When multiple properties are encoded and embedded in the control sample multiple properties may be presented within the user interface. Additionally or alternatively, the metadata information may be added to a data record of the sequence data of the subject specimen or stored in association with the subject specimen.

As a related exemplary action, augmenting sample management can include automatically organizing samples according the detected control sequences of a set of samples S420. The user interface of a genomic computing platform may navigationally organize samples according to shared control sequence metadata. For example, samples of one experiment with a shared experiment number (communicated through information extracted from a control sample) can be automatically grouped. In this way physical application and use of an enhanced spike-in can be used in automatic organization of corresponding sequence data and analysis within a database or computer system.

In addition to data organization, digital grouping and organization of genomic data based on detected control samples can be used in performing automated processing. In one variation, automatically organizing the samples can be used in selectively specifying post-processing of the sequence data. In another variation, automatically organizing the samples can be used in automatically sharing or otherwise setting permissions for access to the genomic data.

As another exemplary action, augmenting sample management may include generating process alerts in response to conditions of the result information and/or control sequence detection S430. One specific example is detection of two or more control sequences with different associated data models may signal cross contamination. An alert can be generated and used to trigger any suitable action within the computing platform. Additionally, omission of an expected control sequence within a group of samples may signal some form of experimental error. Similarly, multiple samples having a shared control sequence when each is expected to have a unique control sequence may signal another form of experimental error.

Accordingly, the method may include a variation that initially includes establishing a data association of a genomic sample and a control sample (e.g., a barcode identifier in the information metadata of the control sample). Establishing a data association may occur during an initial stage of processing the genomic sample. For example, when first adding a genomic sample with a control sample, the experimenter may enter identifiers of the genomic sample and an identifier of the genomic sample. This may be achieved through scanning of a physical barcode, manual entry or through any suitable entry process. With this association, the method can include detecting an anomaly in processing a genomic sample based on detection of the result information and the association with the control sample and generating an alert. This can function to enable flagging cross contamination when detecting a second control sample not associated or similarly, detecting the control sample in a second different genomic sample. The anomaly may include detecting presence of a second, unexpected control sample in association with the genomic sample.

In one variation, experimental equipment can be integrated with or be in communication with the genomic computing platform. Augmenting sample management may include altering state and/or operation of the experimental equipment according to the information from the control sequence S440. For example, sample handling machinery can redirect handling of a genetic sample in response to the information from the control sample. Other types of experimental equipment that can alter operation partially in response to information extracted from a control sample may include sequencing machinery, robotic lab equipment, and/or any suitable automated or semi-automated experimental device or machine.

In one preferred implementation, a genomic computing system generates a report of positive controls, negative controls, and/or cross-contamination. Such information may additionally be reported alongside internal control information.

As another exemplary action, augmenting sample management may include estimating concentration or quantity of detected sequences based on relative detection of a control sequence S450. In some implementations, a computing platform can be configured to have an expected quantity of control sample in a measured genetic sample. This quantity may be based on the amount of enhanced spike-in added to a manufactured kit or device. This quantity may alternatively be specified by a person or machine administering an experiment. The number of reads of the control sequence may be used to estimate the concentration of other detected samples. Preferably, the quantity estimated is generated and reported after calculating a specimen quantity estimate based on the ratio of expected control sequence quantity and measured control sample quantity.

In another exemplary action, augmenting sample management may include processing positive control analysis for the genomic sample S460. Processing a positive control analysis is preferably initiated in response to a detected instance of a control sequence that is registered and configured as a positive control. Processing positive control analysis may include generating a positive control report, which may include reporting at least a contamination level of the positive control. The number and quantity of genetic sequence elements measured in the positive control that are not expected to be found are preferably classified as a contaminant and can be reported. Performing a positive control report may additionally or alternatively include calculating protocol and/or measurement bias and reporting the bias. Evaluation of the positive control and analyzing the sequencing data from the positive control may be used in characterizing type of errors and patterns.

In a similar manner, augmenting sample management may include processing negative control analysis for the genomic sample, which can be triggered when a detected instance of a control sequence that is registered and configured as a negative control. Alternatively, it may be configured within the computing system such that detection of a genomic sample below a threshold of genetic contents is automatically classified as a negative control and then negative control report is generated.

In one preferred variation of the method, multiple different types of control sequences are specified such that there is an internal control and at least a positive control. Variations may additionally support a specified negative control and/or multiple types of internal controls or positive controls. Each of these may be individually detected in different genomic samples (or at times in the same genomic sample) and used in augmenting sample management.

In one example, an implementation of the method for multiple controls may include specifying a first genetic control sequence and a second control sequence, wherein the second control sequence is an organic control sequence and registering the first genetic control sequence as a internal control in a genetic database and registering the second genetic control sequence as a positive control in a genetic database. Then, in association with a first genomic sample, detecting a detected instance of the first control sequence in a collection of genetic data collected for the genomic sample; and augmenting sample management of the first genomic sample within the computing system by presenting identification of resulting information of the control sequence based on an identifier of the internal control sequence (e.g., embedded information or associated metadata). And at another time, in association with a second genomic sample, detecting a detected instance of the second control sequence in a collection of genetic data collected for the second genomic sample; and augmenting sample management of the second genomic sample within the computing system by processing positive control analysis of the second genomic sample. Such combined usage of control sequences within a computing system can be used across any suitable combination of types of control sequences. In some cases, instances of two or more different control sequences may be detected in the same genomic sample and both may be used in augmenting sample management.

The systems and methods of the embodiments can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

Accordingly, a machine-readable storage medium comprising instructions that, when executed by one or more processors of a machine, cause the machine to perform operations that include specifying a control sequence, applying use of a control sample that is generated from the control sequence, detecting the control sequence in sequencing results for a subject sample, and augmenting sample management within the computing system according to the detected control sequence. Any of the suitable variations of the method or system can additionally be directed by the instructions. Similarly, a system for applying the user of enhanced controls with a genomic computing platform can include a processor with a machine-readable storage medium comprising instructions configured to specify a control sequence, optionally apply use of a control sample that is generated from the control sequence, detect the control sequence in sequencing results for a subject sample, and/or augment sample management within the computing system according to the detected control sequence. Any of the suitable variations of the method or system can additionally be directed by the instructions or configured for the system.

As a person skilled in the art will recognize from the previous detailed description and from the figures and claims, modifications and changes can be made to the embodiments of the invention without departing from the scope of this invention as defined in the following claims.

We claim:

1. A method for genomic sample processing in a computing system comprising:
specifying a genetic control sequence;
registering the genetic control sequence to a genetic database;
in association with a genomic sample, detecting a detected instance of the control sequence in a collection of genetic data collected for the genomic sample; and
augmenting sample management of the genomic sample within the computing system according to the detected instance control.

2. The method of claim 1, wherein specifying the genetic control sequence comprises encoding information into the genetic control sequence through an error-correcting code; wherein detecting the detected instance of the control sequence comprises decoding the control sequence and obtaining result information; and wherein augmenting sample management is at least partially based on the result information.

3. The method of claim 2, wherein encoding information into the genomic control sequence comprises encoding the information into the genomic control sequence through an error correcting code using a repetition code.

4. The method of claim 3, wherein the repetition code is a binary repetition code of length three.

5. The method of claim 3, wherein the repetition code is a Hamming code.

6. The method of claim 2, wherein encoding information into the genomic control sequence comprises:
generating a template binary sequence,
transforming the information into a base four data message,
encoding the base four data message with a repetition code,
applying a bitwise exclusive-or operator to the template binary sequence and the encoded sequence, and
outputting the genomic control sequence yielded from successfully encoding the information.

7. The method of claim 6, wherein the repetition code is a Hamming code.

8. The method of claim 6, wherein encoding information into the genomic control sequence comprises: after applying the bitwise XOR operation, validating a set of control conditions for the resulting control sequence, and, if a control condition is not satisfied, generating an updated control sequence by encoding the information using a different random template binary sequence.

9. The method of claim 2, wherein decoding the control sequence thereby obtaining result information comprises:
accessing a template binary sequence,
applying a bitwise exclusive-or operator to the template binary sequence and the detected control sequence,
decoding the repetition encoding, and
converting from base four representation thereby yielding the result information.

10. The method of claim 2, further comprising obtaining a control sample that exhibits a genetic signature based on the control sequence.

11. The method of claim 10, wherein obtaining a control sample based on the control sequence comprises synthesizing the control sample.

12. The method of claim 10, wherein obtaining a control sample based on the control sequence comprises transmitting a synthesis request that specifies the control sequence as a requested control sample.

13. The method of claim 10, further comprising producing a sample collection device prepared with the control sample, wherein the sample collection device is used in association with the genetic sample.

14. The method of claim 2, further comprising establishing an association between the genomic sample and a control sample in the computing system, detecting an anomaly in processing a genomic sample based on detection of the result information and the association with the control sample, and generating an alert.

15. The method of claim 1, wherein the genetic control sequence is an organic control sequence.

16. The method of claim 15, wherein the genetic control sequence is registered as a positive control; and wherein augmenting sample management of the genomic sample comprises processing the genomic sample as a positive control and automatically reporting at least the contamination level of the positive control.

17. The method of claim 1, wherein augmenting sample management of the genomic sample within the computing system comprises: setting a data association of the result information and the collection of genetic data collected for the genomic sample; and presenting the result information in a user interface.

18. The method of claim 17, wherein the genetic control sequence is a synthetic control sequence; and further comprising:
specifying at least a second control, wherein the second control sequence is an organic control sequence and at the computer database of the computing system, registering the genetic control sequence; and
in association with a second genomic sample, detecting a detected instance of the second control sequence in a second collection of genetic data collected for the second genomic sample;
augmenting sample management of the second genomic sample within the computing system according to the detected instance of the second control sequence, which comprises processing the second genomic sample as a positive control.

19. The method of claim 1, wherein augmenting sample management of the genomic sample within the computing system comprises generating a process alert in the computing system in response to conditions of the result information.

20. The method of claim 1, wherein augmenting sample management of the genomic sample within the computing system comprises altering operation of experimental equipment according to the result information.

21. The method of claim 1, where augmenting sample management of the genomic sample within the computing system comprises estimating a quantity of detected sequences based on relative detection of a control sequence.
